(19) United States
(12) Patent Application Publication
(10) Pub. No.: US 2026/0306732 A1
(43) Pub. Date: Oct. 8, 2026

(54) CLEANING DEVICE FOR PRIVATE PARTS

(71) Applicant: Shenzhen Hengnengda Technology Co., Ltd, Shenzhen (CN)

(72) Inventors: Haihui Xing, Shenzhen (CN); Zhixuan Xing, Shenzhen (CN)

(21) Appl. No.: 19/636,901

(22) Filed: Apr. 1, 2026

(30) Foreign Application Priority Data
Apr. 2, 2025 (CN) 202520618466.1

(51) Int. Cl. A61M 3/02; A61M 39/24; A61M 39/00

(52) U.S. Cl. CPC A61M 3/0258; A61M 3/0279; A61M 39/24; A61M 2039/0009; A61M 2205/3327; A61M 2205/50; A61M 2205/8206

(57) ABSTRACT

A cleaning device for private parts is provided, a cleaning rod is rotatably mounted on a housing, and a front cover is mounted on the housing and configured to engage with a water bottle. The front cover is provided with a water inlet and an air inlet, and the air inlet is internally provided with a one-way valve. A water pump, a main control board, and a battery are disposed in the housing, where the main control board is electrically connected to the water pump and the battery, and the water pump is connected to the water inlet. A through hole of the cleaning rod communicates with an internal pipeline of the cleaning rod, the internal pipeline is connected to a water outlet, and a water delivery pipeline of the water pump communicates with the through hole. The cleaning rod is rotatably storable, unfoldable, and adjustable in angle.

### CROSS REFERENCE TO RELATED APPLICATIONS

The present application claims the benefit of Chinese Patent Application No. 202520618466.1 filed on Apr. 2, 2025, the contents of which are incorporated herein by reference in their entirety.

### TECHNICAL FIELD

The present disclosure relates to the technical field of cleaning devices for female private parts, and in particular to a cleaning device for private parts.

### BACKGROUND

Private parts of women are sensitive body areas, and cleanliness of the private parts is closely associated with women’s physical and mental health.

To facilitate women’s cleaning of their private parts during travel or outdoors, manufacturers have developed corresponding cleaning devices.

However, most cleaning devices in the prior art are relatively large in size and are only fixedly disposed in fixed areas, such that it is inconvenient for users to carry. For example, a cleaning device includes a water storage cylinder, a water pump, a water delivery pipeline, and a spray head, and the cleaning device is inevitably bulky and heavy due to the design of the water storage cylinder, such that the cleaning device is inconvenient to carry.

In order to solve the above problems, some manufacturers have placed water storage cylinders externally or designed external water inlets to facilitate carrying by users, but the external water storage cylinders and the external water inlets require users to fetch water manually, thereby resulting in inconvenience in use. Furthermore, cleaning devices in the prior art have the deficiency of privacy leakage. For example, when there is little or no water in the water storage cylinder and the water delivery pipeline, the water pumping action of the water pump generates a lot of noise, which not only brings poor user experience but also may cause privacy leakage of users. When a user holds the spray head of the cleaning device for cleaning, problems such as water splashing everywhere and difficult angle control are prone to occur.

Therefore, there is an urgent need for a cleaning device for private parts that is capable of solving one or more of the above problems.

### SUMMARY

In order to solve one or more problems in the prior art, the present disclosure provides a cleaning device for private parts. The technical solution adopted by the present disclosure to solve the above technical problems is as follows: a cleaning rod is rotatably mounted at a tail end of a housing of the cleaning device, a front cover is mounted at a head end of the housing, the front cover is configured to engage with a water bottle, the front cover is provided with a water inlet and an air inlet, the water inlet and the air inlet spatially correspond to a mouth of the water bottle, and the air inlet is internally provided with a one-way valve;

a water pump, a main control board, and a battery are disposed in the housing, the main control board is electrically connected to the water pump and the battery, and a water inlet end of the water pump is connected to the water inlet; and

the cleaning rod is provided with a through hole, the through hole communicates with a first end of an internal pipeline of the cleaning rod, a second end of the internal pipeline is connected to a water outlet, and a water delivery pipeline of the water pump communicates with the through hole.

In some embodiments, a first sensor is disposed on the water delivery pipeline or the housing, the first sensor is configured to detect whether water exists in the water delivery pipeline, and the first sensor is electrically connected to the main control board; and

when the first sensor is disposed on the housing, the first sensor is arranged to surround the water delivery pipeline.

In some embodiments, at least one electrode sheet and/or a second sensor are/is disposed on an end face of the front cover facing away from the water bottle, the electrode sheet and the second sensor are electrically connected to the main control board, and the main control board detects whether water exists in the front cover through the electrode sheet or the second sensor.

In some embodiments, a first end of the cleaning rod is rotatably connected to a tail end of the housing, a guide groove surrounding a rotating shaft is formed at the first end of the cleaning rod, a butt-joint protrusion engaged with the guide groove is disposed at the tail end of the housing, and the butt-joint protrusion moves along the guide groove when the cleaning rod rotates.

Further, a limiting recess is formed on a side of the guide groove, the limiting recess is engaged with the butt-joint protrusion, and the limiting recess limits rotation of the cleaning rod by limiting the butt-joint protrusion.

In some embodiments, the first end of the cleaning rod is rotatably connected to the tail end of the housing, rotating shafts are disposed on two sides of the first end of the cleaning rod, a support frame is disposed at the tail end of the housing, the rotating shafts are rotatably mounted on the support frame, and the through hole penetrates the rotating shaft on a first side to connect with the water delivery pipeline.

Further, a sealing ring is disposed between the water delivery pipeline and the rotating shaft on the first side.

Further, the housing is provided with a detachable rear housing, the rear housing is fitted over the tail end of the housing, the rear housing is connected to the support frame, and the rear housing limits the rotating shafts in the support frame.

In some embodiments, a storage slot is formed on the housing, and the storage slot is configured to store the cleaning rod.

In some embodiments, a spray head is disposed at the water outlet of the cleaning rod.

The present disclosure has the following technical effects: the front cover of the cleaning device is conveniently butt-jointed with a commercially available mineral water bottle to fix the water bottle, such that a user does not need to specially carry a dedicated water storage bottle, thereby facilitating use outdoors and during travel; the cleaning device extracts water in the water bottle through the water pump and conveys the water to the cleaning rod to achieve the purpose of rinsing and cleaning, where the cleaning rod conveniently rotates for folding and unfolding, and the user can rotate the cleaning rod to achieve better use at a proper angle; the first sensor is arranged to detect water in the water delivery pipeline of the water pump, and the main control board controls the rotational speed of the water pump according to the amount of water, where the rotational speed decreases when the water is reduced until the water pump stops working, such that noise generated when the cleaning device works is reduced, thereby enhancing the user experience and preventing privacy leakage; the first electrode sheet and the second electrode sheet are arranged to achieve capacitive detection of whether water exists, and the main control board controls the rotational speed of the water pump accordingly; and the two detection methods may be used in combination to improve detection precision, or either of the two detection methods may be used separately.

### BRIEF DESCRIPTION OF THE DRAWINGS

FIG. 1 is a schematic view of the present disclosure.

FIG. 2 is a schematic view of connection between a cleaning rod and a housing of the present disclosure.

FIG. 3 is an internal schematic view of the present disclosure.

FIG. 4 is a schematic view of a front cover of the present disclosure.

FIG. 5 is a schematic view of a cleaning rod of the present disclosure.

### REFERENCE NUMERALS IN THE FIGURES

1-housing; 10-rear housing; 100-butt-joint protrusion; 11-storage slot; 12-support frame; 2-front cover; 20-water inlet; 21-air inlet; 22-first electrode sheet; 23-second electrode sheet; 3-cleaning rod; 30-through hole; 300-rotating shaft; 301-guide groove; 302-limiting recess; 31-internal pipeline; 32-water outlet; 4-water pump; 40-water delivery pipeline; 41-butt-joint pipe; 5-battery; 6-main control board; 7-sealing ring; 8-first sensor; and 9-water bottle butt-joint direction.

### DETAILED DESCRIPTIONS OF THE EMBODIMENTS

In order to enable the objectives, features, and advantages mentioned above of the present disclosure to be more apparent and understandable, specific embodiments of the present disclosure will be described in detail below with reference to the accompanying drawings. Numerous specific details are set forth in the following description to facilitate a thorough understanding of the present disclosure. However, the present disclosure may be implemented in many other ways than those described herein, and those skilled in the art may make similar modifications without departing from the spirit of the present disclosure, and therefore the present disclosure is not to be limited by the specific embodiments disclosed below.

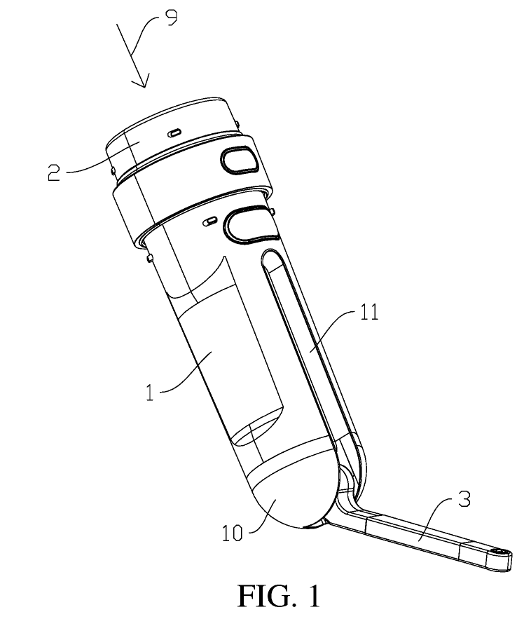

The present disclosure discloses a cleaning device for private parts, and with reference to FIG. 1, a cleaning rod 3 is rotatably mounted at a tail end of a housing 1 of the cleaning device, and a front cover 2 is mounted at a head end of the housing 1;

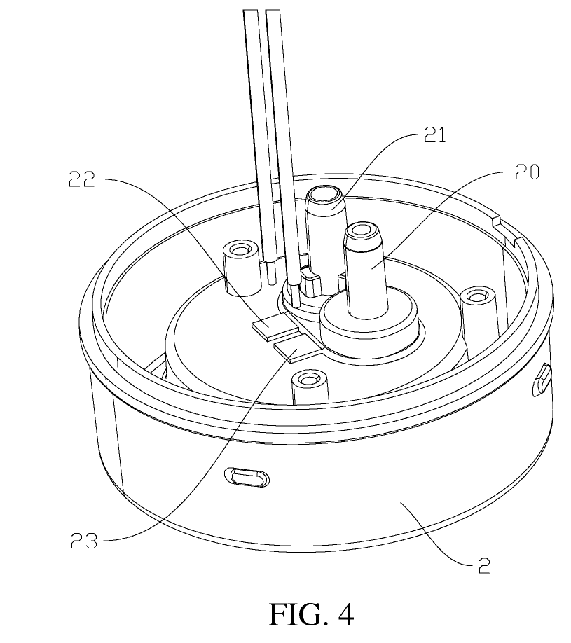

with reference to FIG. 1 and FIG. 4, the front cover 2 is configured to engage with a water bottle, the front cover 2 is provided with a water inlet 20 and an air inlet 21, the water inlet 20 and the air inlet 21 spatially correspond to a mouth of the water bottle, the air inlet 21 is internally provided with a one-way valve, and external air enters the water bottle through the one-way valve and the air inlet 21;

with reference to FIG. 4, a water pump 4, a main control board 6, and a battery 5 are disposed in the housing 1, where the main control board 6 is electrically connected to the water pump 4 and the battery 5, and a water inlet end of the water pump 4 is connected to the water inlet 20; and

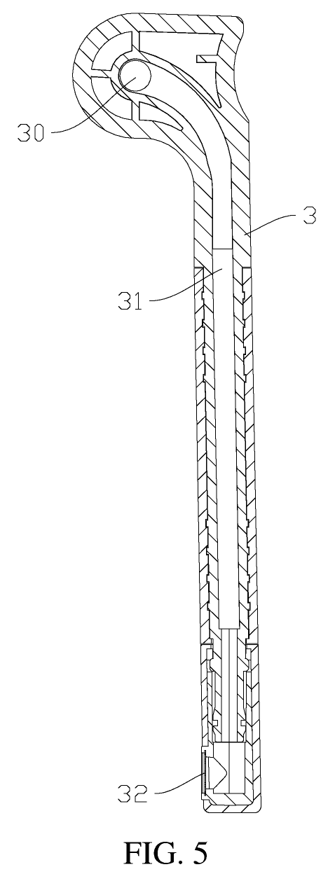

with reference to FIG. 5, the cleaning rod 3 is provided with a through hole 30, the through hole 30 communicates with a first end of an internal pipeline 31 of the cleaning rod 3, a second end of the internal pipeline 31 is connected to a water outlet 32, a water delivery pipeline 40 of the water pump 4 communicates with the through hole 30, and a spray head may be disposed at the water outlet 32.

It should be noted that in use, the mouth of the water bottle is fixed on the front cover 2, and fixing is achieved by means of a threaded connection or a snap-fit clasp. With reference to FIG. 1, when the cleaning device of the present disclosure is in use, the cleaning rod 3 rotates to open and tilt downward, and in this case, a butt-joint port of the front cover 2 is inclined upward, that is, a water bottle butt-joint direction 9 is also inclined, and water in the water bottle naturally flows into the water inlet 20. Furthermore, a storage slot 11 is formed on a side wall of the housing 1, the storage slot 11 is configured to store the cleaning rod 3, and the cleaning rod 3 is stored in the storage slot 11 in a way of rotating.

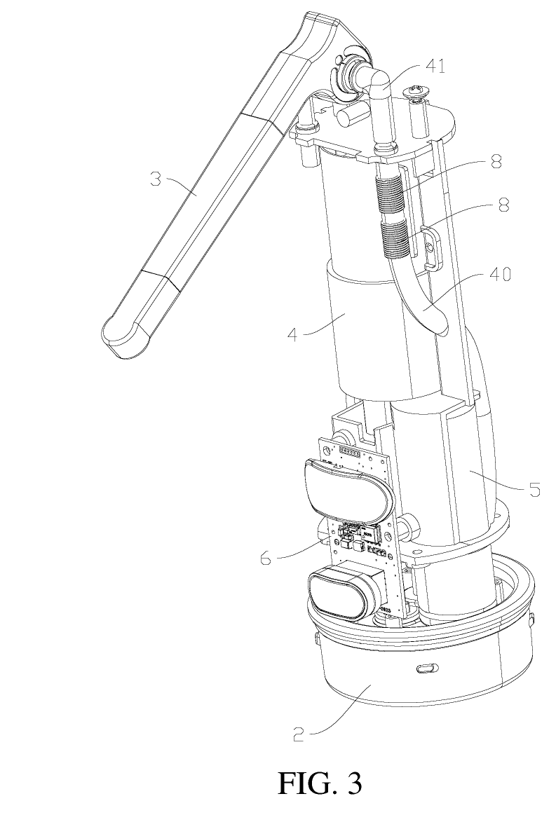

Specifically, with reference to FIG. 3, a first sensor 8 is disposed on the water delivery pipeline 40 or the housing 1, the first sensor 8 is configured to detect whether water exists in the water delivery pipeline 40, and the first sensor 8 is electrically connected to the main control board 6, where the first sensor 8 is a flow sensor, and two first sensors 8 may be arranged; and when the first sensor 8 is disposed on the housing 1, the first sensor 8 is arranged to surround the water delivery pipeline 40. When the first sensor 8 is sleeved on the water delivery pipeline 40, the first sensor 8 is sleeved at an end of the water delivery pipeline 40 close to the cleaning rod 3; and when the first sensor 8 is disposed on the housing 1, the first sensor 8 wraps the end of the water delivery pipeline close to the cleaning rod 3.

The main control board 6 detects a flow rate at the end of the water delivery pipeline 40 close to the cleaning rod 3 through the first sensor 8, and determines whether water exists in the pipeline, and the main control board 6 controls the water pump 4 to speed down until stopping along with reduction of water. When the water is reduced, the rotational speed of the water pump 4 is controlled to decrease to reduce noise generated during water pumping.

Specifically, at least one electrode sheet and/or a second sensor are/is disposed on an end face of the front cover 2 facing away from the water bottle, the electrode sheet and the second sensor are electrically connected to the main control board, and the main control board detects whether water exists in the front cover 2 through the electrode sheet or the second sensor.

With reference to FIG. 5, the electrode sheet may include a first electrode sheet 22 and a second electrode sheet 23, the second sensor may be an ultrasonic sensor, the first electrode sheet 22 and the second electrode sheet 23 are located near the water inlet 20, the first electrode sheet 22 and the second electrode sheet 23 are electrically connected to the main control board 6, and the main control board 6 detects whether water exists in the front cover 2 through the first electrode sheet 22 and the second electrode sheet 23.

The electrode sheets are configured to form a capacitive water level detector. For example, the first electrode sheet 22 and the second electrode sheet 23 form the capacitive water level detector, the main control board 6 detects a capacitance value at the front cover 2 through the electrode sheets to determine whether water exists in the front cover, and the main control board 6 controls the rotational speed of the water pump 4 according to whether water exists and an amount of water, thereby achieving the purpose of reducing noise.

The capacitive water level detector is disposed on an inner wall or an outer wall of the water delivery pipeline to be detected, the electrode sheets (the first electrode sheet and the second electrode sheet) cover a key section of the water delivery pipeline (such as the water inlet of the front cover), and the electrode sheets are made of corrosion-resistant metal (such as gold-plated copper and stainless steel); when water exists in the water delivery pipeline, a dielectric constant (epsilon≈80) of water is far higher than a dielectric constant (epsilon≈1) of air, such that the capacitance value between the electrode sheets significantly increases; the main control board reads the capacitance value in real time through a capacitance measurement circuit (such as a capacitance-to-digital conversion chip/circuit), compares the read capacitance value with a preset capacitance threshold, and finally determines whether water exists, where the preset capacitance threshold (i.e., a water-free capacitance threshold and a water-present capacitance threshold) is set according to experimental data; and output control of the main control board is as follows: if the read capacitance value is lower than the water-free capacitance threshold, the main control board immediately controls the water pump to stop working, and if the read capacitance value is between the water-free capacitance threshold and the water-present capacitance threshold, the main control board controls the water pump to speed down, thereby reducing noise.

In use, the first sensor 8 may be combined with the first electrode sheet and the second electrode sheets to control the water pump to speed down in a more accurate manner, and to control a water pumping speed and noise within a reasonable range; and to reduce the cost, either the first sensor or the electrode sheets may be used, and noise reduction may be achieved when the first sensor and the electrode sheets are used separately.

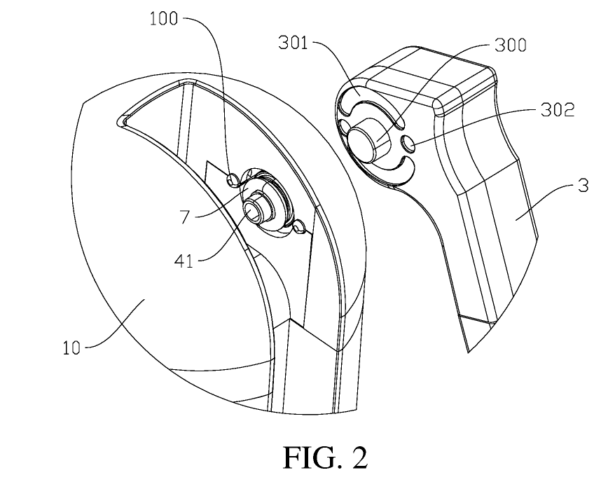

Specifically, with reference to FIG. 1 and FIG. 2, the first end of the cleaning rod 3 is rotatably connected to the tail end of the housing 1, a guide groove 301 surrounding a rotating shaft is formed at the first end of the cleaning rod 3, a butt-joint protrusion 100 engaged with the guide groove 301 is disposed at the tail end of the housing 1, and the butt-joint protrusion 100 moves along the guide groove 301 when the cleaning rod 3 rotates; and

a limiting recess 302 is formed on a side of the guide groove 301, the limiting recess 302 is engaged with the butt-joint protrusion 100, and the limiting recess 302 limits rotation of the cleaning rod 3 by limiting the butt-joint protrusion 100.

Specifically, with reference to FIGS. 1 to 4, the first end of the cleaning rod 3 is rotatably connected to the tail end of the housing 1, rotating shafts 300 are disposed on two sides of the first end of the cleaning rod 3, a support frame 12 is disposed at the tail end of the housing 1, the rotating shafts 300 are rotatably mounted on the support frame 12, and the through hole 30 penetrates the rotating shaft 300 on a first side to connect with the water delivery pipeline 40; and a sealing ring 7 is disposed between the water delivery pipeline 40 and the rotating shaft 300 on the first side, the sealing ring 7 is sleeved at a tail end of the water delivery pipeline 40, the sealing ring 7 provides both a sealing effect and damping for rotation of the cleaning rod 3, and the damping limits the rotation of the cleaning rod 3, and prevents the cleaning rod 3 from shifting due to shaking or slight touch during rotation.

Furthermore, a butt-joint pipe 41 may be a section where the water delivery pipeline 40 is butt-jointed with the through hole 30, the butt-joint pipe 41 is a bent hard pipe, the butt-joint pipe 41 provides a better connecting effect and a better supporting effect, and similarly, a butt-joint head may be disposed at a pipe head of the butt-joint pipe 41. With reference to FIG. 2, when the butt-joint pipe 41 is arranged, the sealing ring 7 is located between the butt-joint pipe 41 and the rotating shaft 300 on the first side, and the sealing ring 7 is sleeved on the butt-joint pipe 41.

With reference to FIGS. 1 to 3, the housing 1 is provided with a detachable rear housing 10, the rear housing 10 is fitted over the tail end of the housing 1, the rear housing 10 is connected to the support frame 12, and the rear housing 10 limits the rotating shafts 300 in the support frame 12.

To sum up, the front cover of the cleaning device is conveniently butt-jointed with a commercially available mineral water bottle to fix the water bottle, such that a user does not need to specially carry a dedicated water storage bottle, thereby facilitating use outdoors and during travel; the cleaning device extracts water in the water bottle through the water pump and conveys the water to the cleaning rod to achieve the purpose of rinsing and cleaning, where the cleaning rod conveniently rotates for folding and unfolding, and the user can rotate the cleaning rod to achieve better use at a proper angle; the first sensor is arranged to detect water in the water delivery pipeline of the water pump, and the main control board controls the rotational speed of the water pump according to the amount of water, where the rotational speed decreases when the water is reduced until the water pump stops working, such that noise generated when the cleaning device works is reduced, thereby enhancing the user experience and preventing privacy leakage; the first electrode sheet and the second electrode sheet are arranged to achieve capacitive detection of whether water exists, and the main control board controls the rotational speed of the water pump accordingly; and the two detection methods may be used in combination to improve detection precision, or either of the two detection methods may be used separately.

In the description of the present disclosure, it is also noted that unless expressly specified and defined otherwise, the terms “mount”, “connected” and “connect”, etc. should be understood in a board sense. For example, the connection may be a fixed connection or a detachable connection; may be a mechanical connection or an electrical connection; may be a direct connection or an indirect connection by using an intermediate medium; or may be intercommunication between two components. For those of ordinary skill in the art, the specific meanings of the terms described above in the present disclosure may be interpreted according to specific circumstances.

The embodiments mentioned above merely express one or more implementations of the present disclosure, and are specifically described in details, but should not be construed as limiting the scope of the present disclosure as a result. It should be noted that for those of ordinary skill in the art, they may also make several modifications and improvements without departing from the concept of the present disclosure, and these modifications and improvements shall fall within the protection scope of the present disclosure. Therefore, the protection scope of the present disclosure shall be governed by the appended claims.

1. A cleaning device for private parts, wherein a cleaning rod is rotatably mounted at a tail end of a housing of the cleaning device, a front cover is mounted at a head end of the housing, the front cover is configured to engage with a water bottle, the front cover is provided with a water inlet and an air inlet, the water inlet and the air inlet spatially correspond to a mouth of the water bottle, and the air inlet is internally provided with a one-way valve;
a water pump, a main control board, and a battery are disposed in the housing, the main control board is electrically connected to the water pump and the battery, and a water inlet end of the water pump is connected to the water inlet; and
the cleaning rod is provided with a through hole, the through hole communicates with a first end of an internal pipeline of the cleaning rod, a second end of the internal pipeline is connected to a water outlet, and a water delivery pipeline of the water pump communicates with the through hole.

2. The cleaning device for private parts according to claim 1, wherein a first sensor is disposed on the water delivery pipeline or the housing, the first sensor is configured to detect whether water exists in the water delivery pipeline, and the first sensor is electrically connected to the main control board; and
when the first sensor is disposed on the housing, the first sensor is arranged to surround the water delivery pipeline.

3. The cleaning device for private parts according to claim 1, wherein at least one electrode sheet and/or a second sensor are/is disposed on an end face of the front cover facing away from the water bottle, the electrode sheet and the second sensor are electrically connected to the main control board, and the main control board detects whether water exists in the front cover through the electrode sheet or the second sensor.

4. The cleaning device for private parts according to claim 1, wherein a first end of the cleaning rod is rotatably connected to a tail end of the housing, a guide groove surrounding a rotating shaft is formed at the first end of the cleaning rod, a butt-joint protrusion engaged with the guide groove is disposed at the tail end of the housing, and the butt-joint protrusion moves along the guide groove when the cleaning rod rotates.

5. The cleaning device for private parts according to claim 4, wherein a limiting recess is formed on a side of the guide groove, the limiting recess is engaged with the butt-joint protrusion, and the limiting recess limits rotation of the cleaning rod by limiting the butt-joint protrusion.

6. The cleaning device for private parts according to claim 1, wherein the first end of the cleaning rod is rotatably connected to the tail end of the housing, rotating shafts are disposed on two sides of the first end of the cleaning rod, a support frame is disposed at the tail end of the housing, the rotating shafts are rotatably mounted on the support frame, and the through hole penetrates the rotating shaft on a first side to connect with the water delivery pipeline.

7. The cleaning device for private parts according to claim 6, wherein a sealing ring is disposed between the water delivery pipeline and the rotating shaft on the first side.

8. The cleaning device for private parts according to claim 6, wherein the housing is provided with a detachable rear housing, the rear housing is fitted over the tail end of the housing, the rear housing is connected to the support frame, and the rear housing limits the rotating shafts in the support frame.

9. The cleaning device for private parts according to claim 1, wherein a storage slot is formed on the housing, and the storage slot is configured to store the cleaning rod.

10. The cleaning device for private parts according to claim 1, wherein a spray head is disposed at the water outlet of the cleaning rod.
